(19) United States
(12) Patent Application Publication
(10) Pub. No.: US 2021/0228833 A1
(43) Pub. Date: Jul. 29, 2021

(54) METHOD AND DEVICE FOR HUMIDIFYING RESPIRATORY GAS

(71) Applicant: Loewenstein Medical Technology S.A., Luxembourg (LU)

(72) Inventors: Ruediger ALSHUT, Karlsruhe (DE); Benno DOEMER, Karlsruhe (DE); Matthias SCHWAIBOLD, Karlsruhe (DE)

(21) Appl. No.: 17/158,207

(22) Filed: Jan. 26, 2021

(30) Foreign Application Priority Data
Jan. 28, 2020 (DE) 102020101950.5

(51) Int. Cl. A61M 16/16; A61M 16/10

(52) U.S. Cl. CPC A61M 16/162; A61M 2205/3331; A61M 2205/3368; A61M 16/1045

(57) ABSTRACT

A ventilator with a respiratory gas unit, and with a humidifier which comprises a heating element and a water container and is designed for coupling to the ventilator, comprising:
at least one sensor which detects one or more parameters of the respiratory gas,
at least one control unit for adjusting the heating power of the heating element at least partially on the basis of the parameter of the respiratory gas.

### CROSS-REFERENCE TO RELATED APPLICATIONS

The present application claims priority under 35 U.S.C. § 119 of German Patent Application No. 102020101950.5, filed Jan. 28, 2020, the entire disclosure of which is expressly incorporated by reference herein.

### BACKGROUND OF THE INVENTION

### 1. Field of the Invention

The present invention relates to a method and a device for humidifying respiratory gas.

### 2. Discussion of Background Information

Respiratory-air humidifiers are typically used in connection with supply of respiratory air in the context of continuous positive airway pressure (CPAP) therapy or bi-level, APAP or home ventilation. They prevent drying-out of the airways, particularly in relatively long ventilation phases. The respiratory-air humidifier generally has a fillable water tank and a heating element. Respiratory-air humidifiers are usually activated in synchrony with the ventilator. Heated and humidified air is thus made available to the patient after a short delay.

Depending on the particular therapy, a variable or constant pressure can be administered to the patient, in order to reduce or eliminate airway closure or to treat acute or chronic respiratory failure.

However, although many patients appreciate the advantages of heated and humidified air, they are unable to get to sleep, precisely because of this heated and humidified air.

Other patients want heated and humidified air to be available to them immediately, i.e. at the start of use of the ventilator. One problem lies in providing heated and humidified air in the event of leakages.

In view of the foregoing, it would be advantageous to have available a respiratory-air humidifier in which the heating and humidifying are improved.

### SUMMARY OF THE INVENTION

The invention provides a ventilator with a respiratory gas unit, and with a humidifier which has a heating element and a water container and is designed for coupling to the ventilator, comprising:
at least one sensor which detects parameters of the respiratory gas,
at least one control unit for setting the heating power of the heating element at least partially on the basis of the parameter of the respiratory gas.

The invention also provides a method for predefining the heating power of a heating element in a ventilator with a humidifier, wherein at least one parameter of the respiratory gas is detected by a sensor, and wherein the predefining of the heating power is done at least partially on the basis of the parameter of the respiratory gas.

According to the invention, the ventilator may also be characterized in that the control unit sets the heating power of the heating element at least partially on the basis of at least one of the following parameters: a pressure of the respiratory gas stream; a flow or volume of the respiratory gas stream; an intentional leakage; an unintentional leakage; a respiratory frequency; an inspiratory tidal volume; an expiratory volume; an I:E ratio; start and end of inspiration; start and end of expiration; a peak flow during inspiration; a peak flow during expiration; ambient temperature; water temperature; air humidity; a starting power or target power of the heating power; a transmission function for the heating power; a volume of the hose; a volume of the user interface; time since start of therapy; measured values of external wireless sensors, e.g. in a smart phone, or of other external data, e.g. weather app in the smart phone.

According to the invention, the ventilator may also be characterized in that the sensor determines the flow of the respiratory gas, and the control unit controls the heating power of the humidifier according to the flow of the respiratory gas.

According to the invention, the ventilator may also be characterized in that the sensor determines the flow of the respiratory gas, and the control unit controls the heating power of the humidifier according to the flow of the respiratory gas and a stored transmission function for the heating power.

According to the invention, the ventilator may also be characterized in that the control unit determines an intentional leakage flow of the respiratory gas and controls the heating power of the humidifier according to the intentional leakage flow of the respiratory gas.

According to the invention, the ventilator may also be characterized in that the control unit determines an unintentional leakage flow of the respiratory gas and controls the heating power of the humidifier according to the unintentional leakage flow of the respiratory gas.

According to the invention, the ventilator may also be characterized in that the control unit determines an average overall flow of the respiratory gas and controls the heating power of the humidifier according to the average overall flow of the respiratory gas.

According to the invention, the ventilator may also be characterized in that the heating power of the humidifier is controlled on the basis of the average overall flow, in such a way that the absolute humidity (water quantity per volume) of the dispensed respiratory gas remains approximately constant.

According to the invention, the ventilator may also be characterized in that the heating power is controlled in order to reduce the drying-out of the mucous membranes of the patient in the event of high leakage and at the same time to prevent a situation where water droplets condense out in the breathing hose in the event of low leakage.

According to the invention, the ventilator may also be characterized in that the control unit controls the heating power of the humidifier such that a constant humidity level in the humidified respiratory gas is achieved over a flow range of from 1 1/min to 300 1/min.

According to the invention, the ventilator may also be characterized in that the flow output by the ventilator is measured via a sensor or determined via an indirect method, for example from the measured pressure and the rotational speed of the fan.

This flow is then averaged by the control unit, preferably over at least one breath or over x (e.g. 2) seconds and independently of the detected respiration phase, so that any trigger errors have no effect on the humidifier regulation.

According to the invention, it may also be possible to omit the digital averaging, and the heating rod can be operated with a highly dynamically fluctuating power—the water of course acts in any case as low-pass filter. The averaging has technical advantages, since the peak load for power supply unit, voltage regulator, heating rod, etc., drops.

According to the invention, the ventilator may also be characterized in that the heating power is modified, in a stepless manner, on the basis of a characteristic map which classifies the required power at certain average overall flows.

According to the invention, the ventilator may also be characterized in that a plurality of discrete steps of heating power are predefinable, wherein in each case at least one characteristic curve per step of the heating power is stored and can be called up.

According to the invention, the ventilator is also characterized in that correction parameters for controlling the heating power are stored, in order to compensate for ambient temperatures or air humidity or water-filling temperatures.

According to the invention, the absolute humidity may also be measured. This serves in particular for measuring the characteristic map. According to the invention, further characteristic maps or correction parameters could also be stored in order to compensate for ambient temperatures/air humidity, water-filling temperatures, etc. The characteristic maps are then read out by the controller and applied.

According to the invention, the ventilator may also be characterized in that it additionally comprises hose heating, wherein the hose heating is controlled according to the flow of overall flow or the leakage.

According to the invention, the ventilator may also be characterized in that the humidifier, upon connection to the ventilator, is coupled to the electronics of the ventilator and is controlled via the ventilator.

According to the invention, the ventilator may also be characterized in that the control unit has at least one ramp module, wherein the ramp module provides a function of the temperature increase in the humidifier, such that the desired humidification or the desired heating power is achieved more quickly.

According to the invention, the ventilator may also be characterized in that the ramp module controls a higher heating power in the first, shorter-lasting phase than in the second, longer phase.

According to the invention, the ventilator may also be characterized in that the ramp module takes account of the temperature in the water container and/or the ambient temperature when controlling the heating power at least in the first phase, and, for this purpose, means for detecting the water temperature are arranged in the humidifier, in order to transmit an actual temperature to the control unit.

If an average or low humidifier step is set, the heating rod heats up very slowly. The constant working point is reached only after 30-90 minutes, so long does the heating phase last. For the patient, when falling asleep, this then feels as if the humidifier is not switched on at all. According to the invention, the ramp module, for example via the function of the temperature increase, now ensures that a very high/the highest heating power is always used for the first x (1-30) minutes, so that the stable working point is reached quickly. For a short time when the humidifying step set is low, for a longer time when the average step is set. The heating rod thus reaches its target temperature after a few minutes. The controller predefines, for example, x minutes ramp module at highest power for each set step of humidification. According to the invention, more intelligent heating phases are also provided which, for example, take into account the temperature of the added water or of the environment.

According to the invention, the ventilator may also be characterized in that the ramp module can predefine different power curves that can be called up or adjusted.

According to the invention, the ventilator may also be characterized in that the ramp module additionally includes a delay module which causes the control unit to adjustably delay the predefining of the heating power of the heating element.

According to the invention, the ventilator may also be characterized in that the delay module causes the control unit to adjustably delay the start of a respiration function.

According to the invention, the ventilator may also be characterized in that the delay module permits a specific time setting in minutes.

According to the invention, the ventilator may also be characterized in that the predefining of different heat stages by the ramp module is realized by means of power regulation, wherein current and voltage at the heating element are detected by a cyclical measurement.

According to the invention, the ventilator may also be characterized in that the power is readjusted by pulse width modulation, so that a constant power output is permitted even when the resistance of the heating element changes.

According to the invention, the ventilator may also be characterized in that the heating element has a temperature-dependent resistance characteristic curve, and, in the ventilator, the actual resistance value is calculated from the measured current and voltage at the heating element.

According to the invention, the ventilator may also be characterized in that the actual resistance value is compared with threshold values in order to identify an empty water container.

According to the invention, the ventilator may also be characterized in that the control unit switches off the heating power if a threshold value is exceeded and signals this state to the user.

According to the invention, the ventilator may also be characterized in that the control unit stores the power actually output to the heating element and makes the values available at at least one interface.

According to the invention, the ventilator may also be characterized in that the control unit has at least one ramp module, wherein the ramp module provides a function of the temperature increase in the humidifier, such that the desired humidification or the desired heating power is achieved more quickly, wherein the ramp module controls a higher heating power, preferably a high or the highest heating power, in a first, shorter-lasting phase than in a second, longer phase, and wherein the sensor determines the flow of the respiratory gas, and the control unit controls the heating power of the humidifier, at least in the second, longer phase, according to the flow of the respiratory gas.

The invention may also be characterized in that the control unit is deactivatable and/or predefines a fixed heating power.

According to the invention, the ventilator may also be characterized in that the heating element has a temperature-dependent resistance characteristic curve, and, in the ventilator, the actual resistance value is calculated from the measured current and voltage at the heating element, wherein the actual resistance value is compared with threshold values in order to identify an empty water container, and wherein the control unit switches off the heating power if a threshold value is exceeded.

The method according to the invention may likewise be characterized in that the parameter of the respiratory gas is the flow of the respiratory gas, wherein the heating power for a first period x of 1 to 30 minutes is at least 75% of the maximum power, and/or the heating element is switched on after an adjustable time delay after the start of respiration.

According to the invention, the control unit may predefine the heating power of the heating element at least partially or temporarily on the basis of the following formula:

H=c_{PL}(θ−0° C.)m_{L}+(r₀+c_{PD}(0−0° C.))m_{w}

where c_{PL} is the specific heat capacity of air

$$( 1.0 \frac{kJ}{kg \cdot K} ) ,$$

θ is the target temperature, r₀ is the evaporation enthalpy of water

$$( 2.6 \frac{MJ}{kg \cdot K} )$$

and c_{PD} is the specific heat capacity of water vapor

$$( 1.9 \frac{kJ}{kg \cdot K} ) .$$

The term (0-0° C.) can also be described as Δ.

For example, a ventilator can provide a continuous positive airway pressure (CPAP) during the entire respiratory cycle of the patient. Bi-level positive airway pressure (Bi-PAP) can provide at least two different pressures in coordination with the inhalation and exhalation efforts made by the patient. In other systems, Auto-PAP (auto-titration positive airway pressure) can regulate the therapeutic pressure on the basis of the degree of respiratory support that the patient needs at a certain point during a breath. The invention can also be used in hospital ventilators, neonatal ventilators or high-flow appliances.

Independently of the particular therapy, these ventilation systems typically comprise at least one respiratory gas unit, as a fan unit or valve unit, and a user interface or mask. A supply hose can connect the respiratory gas unit to the mask, wherein the hose and the mask together define a gas supply line between the respiratory gas unit and the user.

The mask can be configured to be secured relative to the head of the user in such a way that it forms a generally airtight seal with the user's airways (e.g. a seal around the face, the nostrils and/or the mouth). As a result, the respiratory gas unit is able to generate a stream of compressed gas that is dispensed into the airways through the hose.

A humidifier for humidifying the gas delivered from the respiratory gas unit can likewise be provided. Humidifiers typically comprise a heated water reservoir, which contains a water volume with a relatively large surface area. The reservoir is located between the respiratory gas unit and the mask. The respiratory gas from the respiratory gas unit can be conveyed across the water reservoir in which the heated water is located. The respiratory gas takes up moisture and is then made available to the patient at the interface by way of the hose.

Air conditioning: The user sets a power level for the humidifier according to his optimal breathing comfort, which relates to a defined leakage level, preferably with a leaktight mask (only purge flow). When the leakage now increases, the power of the humidifier is increased on the basis of a stored function, e.g., straight line, or on the basis of a characteristic map, since a greater quantity of air per unit of time has to be humidified, and, particularly in the case of mouth leakage, the increased air stream also poses a greater danger of the mucous membranes drying out. By coupling the regulation to the leakage (average air stream), we save on additional sensors such as a humidity sensor at the mask or in the hose. In a preferred design, in addition to the humidifier, the power of the hose heating is also regulated by the same logic, only with other parameterization, since an increased quantity of air also has to be heated in the event of leakage.

The respiratory-air humidifier comprises, for example, a compact apparatus construction which is adapted to the contour of the ventilator. The respiratory-air humidifier comprises a housing bottom part with heating element, a housing top part, and a multifunction middle part of silicone with a sealing support in relation to the housing bottom part and the housing top part.

### BRIEF DESCRIPTION OF THE DRAWINGS

Illustrative embodiments of the invention are set out in the appended drawings, in which:

FIG. 1a and FIG. 1b show the basic set-up of a ventilator which is designed with and without a respiratory-air humidifier;

FIG. 2 shows the humidifying of a respiratory gas at a patient interface in g/m³; and

FIG. 3 shows a ventilator comprising a respiratory gas unit with a humidifier which comprises a heating element and a water container and which is designed for coupling to the ventilator.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

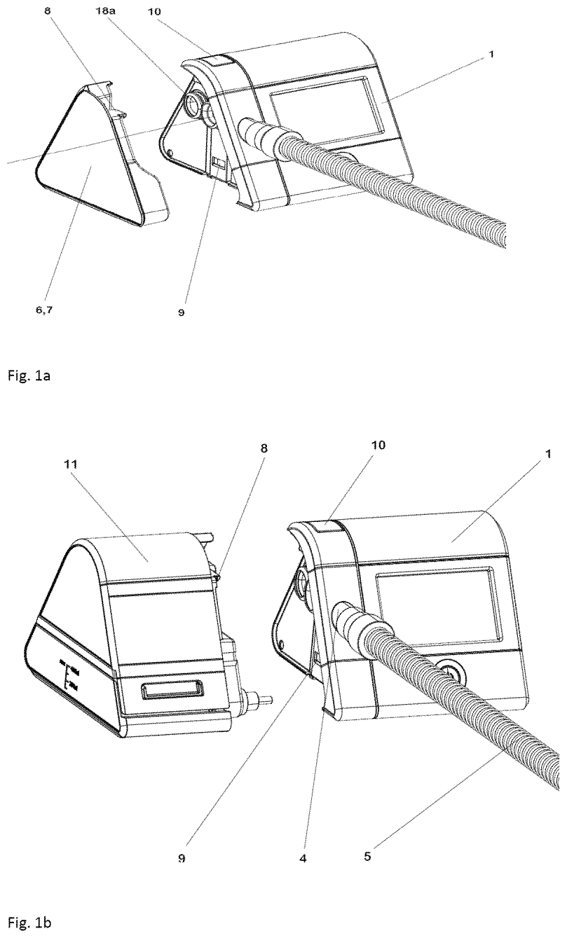

FIG. 1a and FIG. 1b show the basic set-up of a ventilator which is designed with and without a respiratory-air humidifier. The ventilator (1) has an operating panel (2) and a display, for example with touch screen (3). The ventilator is additionally equipped in the apparatus interior with a delivery device for the respiratory gas, e.g., in the form of a respiratory gas pump, a fan or a valve system, and with a controller for the electrical drive. By way of an attachment element (4), a breathing hose (5) is connected, at the end of which is located the interface for the patient, for example in the form of a breathing mask or nasal cannula.

To make available the desired compressed gas stream in the hose, the fan can be part of a flow generator which has a fan housing, which contains a fan wheel or a ventilator. An electric motor, for example a brushless d.c. motor, can rotate the fan wheel or the ventilator during use. As the fan rotates, it sucks respiratory gas (e.g. ambient air) through an inlet of the fan, where the gas is then compressed by the fan and expelled through the outlet. By controlling the speed of rotation of the fan, the pressure of the gas can be controlled in order provide the patient with the desired treatment pressure.

The ventilator can further comprise a controller which, among other duties, can modulate or otherwise control a speed of the motor, for example in order to generate a variable flow rate at a constant pressure. In one embodiment, the controller can contain a microprocessor-based motor controller. All the electrical components can either be fed from a battery and/or an alternating current or direct current source via an electrical cable. The controller can connect other components of the system, as described further herein, electrically to one another, e.g. humidifier and ventilator.

The ventilator can further comprise a pressure sensor. In one embodiment, the pressure sensor is located inside the housing. For example, in the embodiment shown, the pressure sensor is located inside the housing and is connected at or near the outlet. The pressure sensor can generate an electrical signal which is proportional to the actual measured pressure. The pressure signal can then be transmitted via an electrical signal line to the controller. As has been described, the controller can compare the pressure signal with a desired pressure and if necessary can modulate the motor speed at the motor, in order to set the desired pressure. Thus, a desired pressure in the hose/mask can be maintained, irrespective of the expected fluctuations of the flow.

In other embodiments, additional sensors can also be provided, e.g., a pneumotachometer or flow sensor. The pneumotachometer can supply the controller with an electrical signal which is proportional to the instantaneous flow. Other sensors, e.g., temperature, humidity, etc., can likewise be provided and connected electrically to the controller.

Although described as a pneumotachometer, it is also possible to use other devices and/or methods for measuring or estimating the air stream. For example, other embodiments can analyze the speed of the motor or the voltage, the current or the power consumption of the motor.

In order to couple the humidifier (11) to the ventilator (1), the side wall (6) of the ventilator (1), which has an integrated sound damper (7) and is locked with two snap-fit hooks (8) into receptacles (9) in the ventilator (1), is released and replaced by the humidifier (11). For this purpose, the unlocking mechanism (10) on the ventilator (1) simply needs to be activated. By actuation of the unlocking mechanism (10, the receptacles (9), which are connected to each other on a connection rail, are displaced, and the connection to the snap-fit hooks (8) is released. The humidifier (11) likewise has snap-fit hooks (8), which engage in the same receptacles (9) of the ventilator (1).

The housing bottom part (12) has an opening for the heating element (13), which is screwed laterally into the opening of the housing bottom part (12) and is sealed with an O-ring. When the humidifier (11) is attached to the ventilator (1), the heating element (13) is coupled to the electronics of the ventilator (1) and controlled via the ventilator (1).

The interior of the housing bottom part (12) serves to receive the water, and the water level can be read off via filling level indicators.

The humidifier is controlled via the regulation of the ventilator. Here, different operating states can be realized. The heater of the humidifier can pre-heat prior to the operation of the ventilator. Alternatively, the heating of the humidifier can be delayed, i.e., switched on after the start of ventilation. Various power curves can also be realized. For example in the form of a ramp, wherein the power is at first kept very low and then rises abruptly to a high power or else can rise linearly. The pre-selection of different heating stages is likewise possible.

The heating stages can be realized by means of power regulation. Here, current and voltage at the heating element (13) can be detected by a cyclical measurement and, for example, readjusted by a pulse width modulation, so that a constant power output is permitted even in the event of a changing resistance of the heating element.

The controller can analyze data in order to determine various parameters assigned to the operation of the ventilator. For example, respiratory frequency, tidal volume, intentional leakage flow, gas flow, mask leakage flow, inspiratory/expiratory transitions, prediction of the start and/or duration of inspiration phases of subsequent (future) respiratory cycles, operating parameters of the ventilator or of the humidifier. Further parameters relating to operation are, for example, the volume (e.g. length and diameter) of the hose, the intentional mask leakage, the mask dead space, and the desired degree of humidification (predefined by the user). Some of these parameters can be explicitly input, while others can be selected from configurations.

Instead of, for example, inputting explicit dimensions for the hose and/or the mask, it is possible for the user simply to select a hose or mask part number. The controller can then automatically determine an intentional leakage, hose volume and other parameters.

In a configuration like the one in FIG. 1, a relatively large volume of water with a correspondingly large surface area is provided, so that the stream of compressed gas generated by the fan can capture the desired vapor content as it flows across the water in the reservoir. For example, a humidifier may have a reservoir size of 200 to 800 ml and require on average 40 to 500 watt in order to deliver a desired humidity level.

The humidifier can contain a heating element by which a volume of water contained in the reservoir is heated to a desired point at which the water at least partially evaporates. A rigid or flexible conduit can connect an output of the fan of the ventilator to an inlet of the reservoir of the humidifier, while a hose typically connects an outlet of the reservoir to an inlet of the mask. As has been described above, the humidifier can also be adaptable directly to the ventilator.

The hose or the mask or an intermediate piece can have an exhaust air opening. A desired/intentional leakage flow can stream permanently through this exhaust air opening in order to carry away exhalation gases. A stream of compressed gas generated by the fan can stream through the humidifier reservoir, wherein evaporating moisture is entrained therein in order to generate a stream of humidified gas which, finally, is conveyed from the hose to the mask and to the patient.

The user interface or mask is shown in general terms but comprises virtually any interface that effectively seals off a patient (e.g., the face (nose and/or mouth) or inside the nostrils) such that a flow of compressed gas is obtained which is dispensed to the user interface. For example, the user interface could be a face mask, which covers the mouth and/or the nose of the user; a nostril pad; or a combination of such masks. For simplicity, the user interface can be designated hereinafter without limitation also as “mask”.

The hose and the user interface (mask) can together define a feed line which forms a passage that transports the stream of compressed gas from the outlet of the fan to the humidifier and onward to the inlet of the mask and then to the airway or that communicates in some other way with the patient. The feed line as a whole can contain one or more exhaust air openings. It is known that such exhaust air openings make available a so-called “intentional or desired leakage”. An intentional leakage is made available in order to assist the purging of carbon dioxide (with the exhaled respiratory gas) from the feed line during the exhalation phase of each respiratory cycle. In one embodiment, one or more exhaust air openings can be provided. The intentional or desired leakage flow is known or can be calculated for defined flows and pressures.

Automatic detection of an empty water container is permitted by the fact that the heating element (13) has a temperature-dependent resistance characteristic curve, and in the ventilator (1) the actual resistance value is calculated from the measured current and the voltage at the heating element (13) and compared with threshold values. In the case of an empty water container, the heating element (13) will reach a higher temperature than in the case where water is still present. If the threshold value is exceeded, the heating power can be switched off and this state can be signaled to the user.

If the heater of the humidifier already pre-heats prior to the operation of the ventilator, it must be ensured that the water does not condense outside the humidifier. This can be prevented by limiting the pre-heating time in the ventilator. If the operation of the ventilator is not activated within this time, the pre-heating can be automatically discontinued. In some embodiments, the detection can be set so finely that even an incomplete coverage of the heating element with water can be detected via the temperature that is reached and/or via the resistance of the heating element.

In heating pauses, i.e., when heating of the water is not taking place and/or the heating element is not heated, it is also possible, in some embodiments, to determine the water temperature via the resistance of the heating element. For example, the water temperature can be determined on the basis of a temperature-resistance characteristic curve of the heating element. The water temperature that is determined can then be used, for example, by the control unit to estimate whether or when heating can and/or must be carried out again.

In an alternative and/or extended embodiment of the invention, the water temperature determined during the heating pauses and the energy introduced between two heating pauses by the heating element (for example calculated from voltage and current and various material constants and variables of the heating element) can be used to make an at least rough estimate of the filling level and, if appropriate, this can be output on a display. The rough estimate can be given in steps such as “full”, “half full and “empty”, or also as filling percentages (100% to 65%, 65% to 35%, below 35%).

For example, the ventilator and/or the humidifier or the controller/control unit comprises a ramp module, which includes a function of the temperature increase. By way of this function, it is possible, for example in the first x (1 to 30) minutes, to use a very high/the highest heating power (for example at least 75% of the maximum power) so that a stable working point is quickly reached. Depending on the desired/set stage of humidification, the duration of the high heating power, for example, can be adapted either manually or automatically. For example, if a low humidification stage is set, heating is provided at high/maximum power for a shorter time than would be set for higher humidification stages. According to the invention, more intelligent heating phases may also be provided which, for example, take account of the temperature of the water introduced or of the environment.

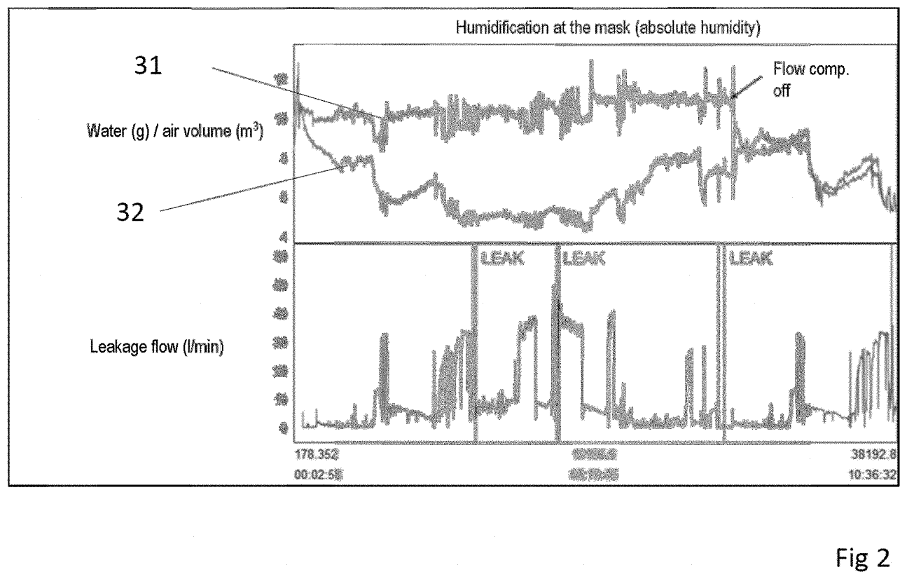

FIG. 2 shows the humidifying of the respiratory gas at the patient interface (here a mask) in g/m³. In In the upper part of the illustration, an upper recording (31) can be seen in which the humidity of the respiratory gas remains relatively constant in the range of 10-11 g/m³. This upper recording has the pre-defining of the heating power, according to the invention, of the heating element (13) at least partially based on the parameter of the respiratory gas, i.e. based on the flow or the leakage or the average overall flow. At the point marked with an arrow, the pre-defining of the heating power of the heating element (13) at least partially based on the parameter of the respiratory gas is switched off. Thereafter, the humidity of the respiratory gas initially drops to 8 and then to 6 g/m³.

The lower recording (32) shows the profile of the humidity of the respiratory gas where the heating power of the heating element (13) is predefined without account being taken of parameters of the respiratory gas. Here, the humidity of the respiratory gas drops from 10 to 5 g/m³.

In the lower part of the illustration, the leakage flow in 1/min is shown in synchrony with the upper profile of the humidity of the respiratory gas. It will be seen that, when the leakage increases, the humidity of the respiratory gas in the lower recording (32) (without the flow compensation according to the invention) falls sharply.

The controller can be configured to simply monitor a rate of the flow (flow or the leakage or the average overall flow) of compressed gas and to automatically modulate the electrical power supplied to the heating element during the respiratory cycle. This process can modulate the quantity of moist, heated vapor that is added proportionally to the stream of compressed gas, in order to maintain a substantially constant target humidity level in the stream of compressed gas (even if the stream of compressed gas changes) during the respiratory cycle and during the treatment. The controller can also analyze current or previous respiratory cycles, in order to predict future respiration/humidification requirements and to adapt the pre-defining of the heating element.

In some illustrative embodiments, the control is carried out on the basis of stored characteristic maps in which a required heating power of the heating element is placed in relation to the flow of the respiratory gas and/or a leakage flow.

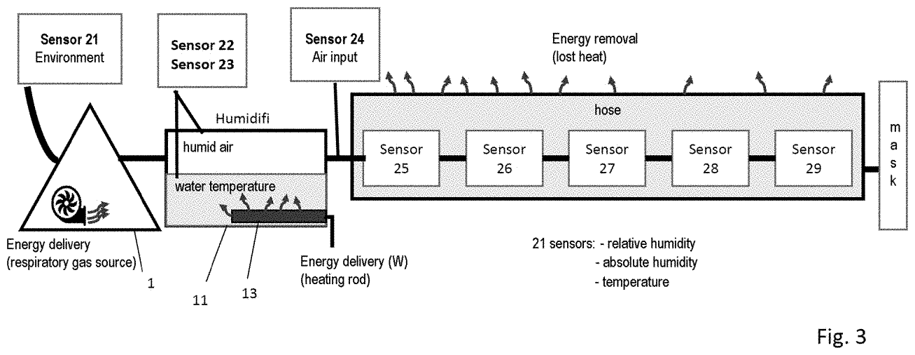

FIG. 3 shows a ventilator having a respiratory gas unit (14) with a humidifier (11) which comprises a heating element (13) and a water container and which is designed for coupling to the ventilator (1), comprising: at least one sensor which detects parameters of the respiratory gas, and at least one control unit (16) for pre-defining the heating power of the heating element (13) at least partially on the basis of the parameter of the respiratory gas or at least partially on the basis of at least one of the following parameters: a pressure of the respiratory gas stream; a flow or volume of the respiratory gas stream; an intentional leakage; an unintentional leakage; a respiratory frequency; an inspiratory tidal volume; an expiratory volume; an I:E ratio; start and end of inspiration; start and end of expiration; a peak flow during inspiration; a peak flow during expiration; ambient temperature; water temperature; air humidity; a starting power or target power of the heating power; a transmission function for the heating power; a volume of the hose; a volume of the user interface; time since start of therapy; measured values of external wireless sensors, e.g. in a smart phone, or of other external data, e.g. weather app in the smart phone.

Different sensors 21 to 29 detect the ambient temperature, the ambient humidity, the humidity in the humidifier, the water temperature in the humidifier, the stream of respiratory gas and the heat losses in the system and the humidity in the mask. Overall, the relative humidity and the absolute humidity are determined. The heating power is changed, for example in a stepless manner, on the basis of a characteristic map which classifies the required power at certain average overall flows.

Several discrete stages of heating power can preferably be predefined, wherein in each case at least one characteristic curve per stage of the heating power is stored and can be called up. The characteristic curves may have been determined based on the layout in FIG. 3.

The heating power is controlled, for example, on the basis of correction parameters that are stored in order to compensate for ambient temperatures or air humidity or water temperatures.

The heating power is also controlled, for example, on the basis of data of a resistance sensor, such that the heating power is reduced at a low water level.

Within the meaning of the invention, humidifiers of the kind described here can humidify respiratory gas in ventilators by using an evaporation device which evaporates water when the device is operated electrically.

The combination of the control based on the respiratory flow and/or leakage flow (intentional, unintentional) with the function of the temperature increase of the ramp module permits comfortable control and humidification of the respiratory gas. If the user/patient expects a high level of humidification of the respiratory air and/or sets this level, the heating power of the heating element is controlled at a high or the highest stage by the function of the temperature increase at the start of the therapy session/the ventilation in a first, relatively short phase (e.g. at most the first 30 minutes). In this way, the water in the humidifier is quickly heated and, as a result, the desired humidification of the respiratory gas is quickly achieved.

When the desired humidification of the respiratory gas or a corresponding water temperature is reached, it is possible, in the second and longer phase, to control the heating power for example on the basis of the overall flow of the respiratory gas and/or on the basis of leakage flows. For this purpose, for example, characteristic maps are stored which place the required heating power in relation to the actual flow or the desired humidification of the respiratory gas.

The figures are provided primarily to aid understanding and are therefore not necessarily true to scale.

Moreover, various structures/components, including but not limited to fastening elements, electrical components (wiring, cables, etc.) and the like, may have been shown schematically or may have been removed from all or some of the views, in order to better illustrate aspects of the depicted embodiments, or where the inclusion of such structures/components is not necessary to the understanding of the various illustrative embodiments described herein.

Where such structures/components are not shown/described in a certain figure, this is not to be interpreted as in any way limiting the scope of the various embodiments.

The term “gas”, as used here, comprises almost every gas or gas/vapor combination. For example, the gas made available by the fan can contain air, oxygen, water vapor or water droplets, medical vapor or medical vapors, and combinations thereof. To simplify the description, the terms “air”, “fluid” and “gas” can be used interchangeably herein. “Compressed gas”, as used herein, relates to gas with a positive pressure in relation to the ambient pressure. “Fan”, as used herein, relates to the majority of appliances or sources that are able to generate a stream of compressed gas.

To sum up, the present invention provides:

1. A ventilator with a respiratory gas unit, and with a humidifier which has a heating element and a water container and is designed for coupling to the ventilator, comprising at least one sensor which detects one or more parameters of the respiratory gas and at least one control unit for predefining the heating power of the heating element at least partially on the basis of the parameter of the respiratory gas.

2. The ventilator of item 1, wherein the control unit sets the heating power of the heating element at least partially on the basis of at least one of the following parameters: a pressure of the respiratory gas stream; a flow or volume of the respiratory gas stream; an intentional leakage; an unintentional leakage; a respiratory frequency; an inspiratory tidal volume; an expiratory volume; an I:E ratio; start and end of inspiration; start and end of expiration; a peak flow during inspiration; a peak flow during expiration; ambient temperature; water temperature; air humidity; a starting power or target power of the heating power; a transmission function for the heating power; a volume of the hose; a volume of the user interface; time since start of therapy; measured values of external wireless sensors, e.g. in a smart phone, or of other external data, e.g. weather app in the smart phone.

3. The ventilator of items 1 or 2, wherein the sensor determines the flow of the respiratory gas, and the control unit controls the heating power of the humidifier according to the flow of the respiratory gas.

4. The ventilator of any one of the preceding items, wherein the sensor determines the flow of the respiratory gas, and the control unit controls the heating power of the humidifier according to the flow of the respiratory gas and a stored transmission function for the heating power.

5. The ventilator of any one of the preceding items, wherein the control unit determines a leakage flow of the respiratory gas and controls the heating power of the humidifier according to the leakage flow of the respiratory gas, the leakage flow being an intentional leakage flow and/or an unintentional leakage flow.

6. The ventilator of any one of the preceding items, wherein the heating power of the humidifier is controlled on the basis of the average overall flow, in such a way that the absolute humidity (water quantity per volume) of the dispensed respiratory gas remains approximately constant, and/or in order to reduce the drying out of the mucous membranes of the patient in the event of high leakage and at the same time to prevent a situation where water droplets condense out in the breathing hose in the event of low leakage.

7. The ventilator of any one of the preceding items, wherein the control unit controls the heating power of the humidifier such that a constant humidity level is achieved in the humidified respiratory gas over a flow range of from 1 1/min to 300 1/min.

8. The ventilator of any one of the preceding items, wherein the flow dispensed by the ventilator is measured via a sensor or determined via an indirect method, for example from the measured pressure and the rotational speed of the fan, and wherein the flow is averaged by the control unit, preferably over at least one breath or over at least 2 seconds and independently of the detected respiration phase, such that any trigger errors have no effect on the humidifier regulation.

9. The ventilator of any one of the preceding items, wherein the heating power is modified in a stepless manner, on the basis of a characteristic map which classifies the required power at certain average overall flows.

10. The ventilator of any one of the preceding items, wherein a plurality of discrete steps of heating power are predefinable, wherein in each case at least one characteristic curve per step of the heating power is stored and can be called up.

11. The ventilator of any one of the preceding items, wherein the ventilator additionally comprises hose heating, the hose heating being controlled according to the flow or overall flow or the leakage.

12. The ventilator of any one of the preceding items, wherein the humidifier, upon connection to the ventilator, is coupled to the electronics of the ventilator and is controlled via the ventilator.

13. The ventilator of any one of the preceding items, wherein the control unit has at least one ramp module, wherein the ramp module provides a function of the temperature increase in the humidifier, such that the desired humidification or the desired heating power is achieved more quickly, the ramp module controlling a higher heating power in a first, shorter-lasting phase than in a second, longer phase.

14. The ventilator of item 13, wherein the ramp module takes account of the temperature in the water container and/or the ambient temperature when controlling the heating power at least in the first phase, and, for this purpose, elements are arranged for detecting the water temperature in the humidifier, in order to transmit an actual temperature to the control unit, wherein the ramp module can predefine different power curves that can be called up or adjusted.

15. The ventilator of item 13 or item 14, wherein the ramp module additionally includes a delay module which causes the control unit to adjustably delay predefining the heating power of the heating element, wherein the delay module permits a specific time setting, for example in minutes, and wherein the delay module causes the control unit to adjustably delay the start of a respiration function.

16. The ventilator of any one of items 13-15, wherein the predefining of different heat stages by the ramp module is realized by means of power regulation, wherein current and voltage at the heating element are detected by a cyclical measurement.

17. The ventilator of any one of the preceding items, wherein the power is readjusted by pulse width modulation, so that a constant power output is permitted even when the resistance of the heating element changes.

18. The ventilator of any one of the preceding items, wherein the control unit is deactivatable and/or predefines a fixed heating power.

19. The ventilator of item 1, wherein the control unit has at least one ramp module, wherein the ramp module provides a function of the temperature increase in the humidifier, such that the desired humidification or the desired heating power is achieved more quickly, wherein the ramp module controls a higher heating power, preferably a high or the highest heating power, in a first, shorter-lasting phase than in a second, longer phase, and wherein the sensor determines the flow of the respiratory gas, and the control unit controls the heating power of the humidifier, at least in the second, longer phase, according to the flow of the respiratory gas.

20. The ventilator of any one of the preceding items, wherein the heating element has a temperature-dependent resistance characteristic curve, and, in the ventilator, the actual resistance value is calculated from the measured current and voltage at the heating element, wherein the actual resistance value is compared with threshold values in order to identify an empty water container, and wherein the control unit switches off the heating power if a threshold value is exceeded.

21. A method for predefining the heating power of a heating element in a ventilator with a humidifier, wherein at least one parameter of the respiratory gas is detected by a sensor, and wherein the predefining of the heating power is done at least partially on the basis of the parameter of the respiratory gas.

22. The method of item 21 for predefining the heating power of a heating element in a ventilator with a humidifier, wherein the parameter of the respiratory gas is the flow of the respiratory gas, wherein the heating power for a first period x of 1 to 30 minutes is at least 75% of the maximum power, and/or the heating element is switched on after an adjustable time delay after the start of respiration.

What is claimed is:

1. A ventilator, wherein the ventilator comprises a respiratory gas unit and a humidifier comprising a heating element and a water container and being configured for coupling to the ventilator, and comprising:
at least one sensor (which detects one or more parameters of the respiratory gas,
at least one control unit for predefining a heating power of the heating element at least partially on the basis of the one or more parameters of the respiratory gas.

2. The ventilator of claim 1, wherein the control unit sets the heating power of the heating element at least partially on the basis of at least one of the following parameters: a pressure of the respiratory gas stream; a flow or volume of the respiratory gas stream; an intentional leakage; an unintentional leakage; a respiratory frequency; an inspiratory tidal volume; an expiratory volume; an I:E ratio; start and end of inspiration; start and end of expiration; a peak flow during inspiration; a peak flow during expiration; ambient temperature; water temperature; air humidity; a starting power or target power of the heating power; a transmission function for the heating power; a volume of a hose; a volume of a user interface; time since start of therapy; measured values of external wireless sensors or of other external data.

3. The ventilator of claim 1, wherein the sensor determines a flow of the respiratory gas, and the control unit controls the heating power of the humidifier according to the flow of the respiratory gas.

4. The ventilator of claim 1, wherein the sensor determines a flow of the respiratory gas, and the control unit controls the heating power of the humidifier according to the flow of the respiratory gas and a stored transmission function for the heating power.

5. The ventilator of claim 1, wherein the control unit determines a leakage flow of the respiratory gas and controls the heating power of the humidifier according to the leakage flow of the respiratory gas, the leakage flow being an intentional leakage flow and/or an unintentional leakage flow.

6. The ventilator of claim 1, wherein the heating power of the humidifier is controlled on the basis of an average overall flow, in such a way that an absolute humidity (water quantity per volume) of dispensed respiratory gas remains approximately constant, and/or in order to reduce a drying out of mucous membranes of a patient in the event of high leakage and at the same time to prevent a situation where water droplets condense out in a breathing hose in the event of low leakage.

7. The ventilator of claim 1, wherein the control unit controls the heating power of the humidifier such that a constant humidity level is achieved in the humidified respiratory gas over a flow range of from 1 1/min to 300 1/min.

8. The ventilator of claim 1, wherein a flow dispensed by the ventilator is measured via a sensor or determined via an indirect method, and wherein the flow is averaged by the control unit, such that any trigger errors have no effect on humidifier regulation.

9. The ventilator of claim 1, wherein the heating power is modified in a stepless manner, on the basis of a characteristic map which classifies a required power at certain average overall flows.

10. The ventilator of claim 1, wherein a plurality of discrete steps of heating power are pre-definable, and wherein in each case at least one characteristic curve per step of the heating power is stored and can be called up.

11. The ventilator of claim 1, wherein the ventilator additionally comprises hose heating, the hose heating being controlled according to a flow or overall flow or a leakage.

12. The ventilator of claim 1, wherein the humidifier, upon connection to the ventilator, is coupled to electronics of the ventilator and is controlled via the ventilator.

13. The ventilator of claim 1, wherein the control unit comprises at least one ramp module, the ramp module providing a function of a temperature increase in the humidifier, such that a desired humidification or a desired heating power is achieved more quickly, and wherein the ramp module controls a higher heating power in a first, shorter-lasting phase than in a second, longer phase.

14. The ventilator of claim 13, wherein the ramp module takes account of a temperature in the water container and/or an ambient temperature when controlling the heating power at least in the first phase, and, for this purpose, elements are arranged for detecting a water temperature in the humidifier, in order to transmit an actual temperature to the control unit, and wherein the ramp module can predefine different power curves that can be called up or adjusted.

15. The ventilator of claim 13, wherein the ramp module additionally comprises a delay module which causes the control unit to adjustably delay predefining the heating power of the heating element, the delay module permitting a specific time setting and causing the control unit to adjustably delay a start of a respiration function.

16. The ventilator of claim 15, wherein a predefining of different heat stages by the ramp module is realized by power regulation, wherein current and voltage at the heating element are detected by a cyclical measurement.

17. The ventilator of claim 16, wherein power is readjusted by pulse width modulation, so that a constant power output is permitted even when a resistance of the heating element changes.

18. The ventilator of claim 1, wherein the control unit comprises at least one ramp module, wherein the ramp module provides a function of a temperature increase in the humidifier, such that a desired humidification or a desired heating power is achieved more quickly, wherein the ramp module controls a higher heating power in a first, shorter-lasting phase than in a second, longer phase, and wherein the sensor determines a flow of the respiratory gas, and the control unit controls the heating power of the humidifier, at least in the second, longer phase, according to the flow of the respiratory gas.

19. The ventilator of claim 1, wherein the heating element has a temperature-dependent resistance characteristic curve, and, in the ventilator, an actual resistance value is calculated from a measured current and voltage at the heating element, the actual resistance value being compared with threshold values in order to identify an empty water container, and the control unit switching off the heating power if a threshold value is exceeded.

20. A method for predefining the heating power of a heating element in a ventilator with a humidifier, wherein at least one parameter of a respiratory gas is detected by a sensor, and wherein the predefining of the heating power is done at least partially on the basis of the parameter of the respiratory gas.
